(19) United States
(12) Patent Application Publication
(10) Pub. No.: US 2025/0153150 A1
(43) Pub. Date: May 15, 2025

(54) HETEROGENEOUS CATALYST BASED ON MOLYBDENUM OXIDE AND ITS USE IN BIOMASS TRANSFORMATION PROCESSES

(71) Applicant: Instituto Mexicano del Petróleo, Mexico City (MX)

(72) Inventors: Diego Gerardo Valencia Mendoza, Mexico City (MX); Rosa Isela Conde Velasco, Mexico City (MX); Jorge Arturo Aburto Anell, Mexico City (MX); María Teresa Gómez Pérez, Mexico City (MX)

(21) Appl. No.: 18/941,563

(22) Filed: Nov. 8, 2024

(30) Foreign Application Priority Data
Nov. 9, 2023 (MX) MX/A/2023/013281

(51) Int. Cl. B01J 23/883; B01J 6/00; B01J 21/08; B01J 23/881; B01J 23/885; B01J 37/02; C07C 5/52

(52) U.S. Cl. CPC B01J 23/883; B01J 6/001; B01J 21/08; B01J 23/881; B01J 23/885; B01J 37/0201; C07C 5/52; C07C 2523/881; C07C 2523/883; C07C 2523/885

(57) ABSTRACT

The process for synthesizing heterogeneous sulfur-free catalysts based on molybdenum oxide, as well as their use in biomass transformation processes, constitutes the central part of this invention. These materials were shown to be active and selective in three biomass transformation processes of industrial importance, namely: (i) hydrodeoxygenation of fatty acids, (ii) hydrogenation and (iii) dehydrogenation of terpenes. The synthesis conditions of these catalysts allow to obtain 100% conversion of fatty acids and the production of linear hydrocarbons without oxygen in their structure above 95%. The products generated using the heterogeneous catalysts of this invention have great relevance in various industries such as energy, chemicals, oil, among others. The operating conditions of these catalysts were also optimized to achieve their application in the most favorable conditions, reducing reaction time, temperature and pressure. The versatility of these materials, their simple handling, the use of inexpensive metals and in small quantities facilitate their use in large-scale industrial processes.

### TECHNICAL FIELD OF INVENTION

The present invention is related to the use of new materials as heterogeneous catalysts in hydrogenation and dehydrogenation processes of compounds present in vegetable oils and animal fats. Specifically, the materials within this invention show very good performance as catalysts in the hydrodeoxygenation (HDO) reactions of fatty acids, as well as the hydrogenation and dehydrogenation of terpenes.

### BACKGROUND TO THE INVENTION

The search for new sources of energy, which can meet the needs of contemporary societies and that are sustainable, has led to the development of new technology and processes that impact on the environment is minimal or less negative in the short term. In this sense, biomass processing is attractive for generating products like those obtained from non-renewable sources such as petroleum. In this scenario, the molecules or chemical products will have very similar properties and uses with the current infrastructure, although they would come from very different sources, which allow them to be called drop-in. On the one hand, it is possible to replace or complement oil as a raw material, with biomass, which is renewable and available from different industries, as by-products or waste. Making this transition also poses many scientific and technological challenges, since biomass contains large amounts of oxygenated compounds, the presence of water and chemical compounds that are incompatible with the existing infrastructure.

There are several uses for the products of the HDO reactions of derivatives of vegetable oils and animal fats, since oxygen-free hydrocarbons would in principle replace those currently used and obtained from the refining of petroleum. For example, HDO products in palm oil give linear hydrocarbons in the range of C₁₅-C₂₀, which is a similar fraction of the compounds present in diesel, for this reason we can speak about green or renewable diesel. On the other hand, this fraction is free of aromatic compounds and is mostly composed of linear hydrocarbons, which would make a high-quality diesel, even an additive that would improve the cetane number. This product as an additive has a much higher added value, which makes it attractive to be added to fossil diesel, which would also reduce the carbon footprint of the final fuel. In addition, linear plant-based hydrocarbons have great appeal in another area as flow improvers in oil well drilling by improving the viscosity of the oil being extracted. Another area is lubricants, in which the base is made up of linear hydrocarbons, which are typically obtained from an exhaustive refining of the fractions obtained from petroleum. The exchange of these bases for mixtures obtained from the processing of biomass would make lubricants sustainable from the point of view of the raw material, as well as the less drastic process that provides a high amount of linear hydrocarbons.

The aviation sector is also among those that foresee a near horizon where its fuels contain an important renewable component. In this sense, biojet fuel or green jet fuel aims to provide a fuel with properties like those of fossil fuel, so that aircraft contribute to the reduction of greenhouse gas emissions. Hydrodeoxygenation of fatty acid derivatives followed by hydroisomerization processes is an alternative to produce branched paraffins as required in jet fuel. On the other hand, terpenoids, and monoterpenes, such as limonene and its derivatives are also envisioned as potential precursors of biojet fuel, in an effort to have alternatives for the advanced aviation fuels sector. However, the direct combustion of limonene in combustion engines has negative effects due to the formation of rubbers. In this sense, the total hydrogenation of limonene to produce p-mentane is an attractive alternative for this cyclic hydrocarbon to be converted into advanced aviation fuel [Energy Environ. Sci., 2019, 12, 807-824]. Total hydrogenation of limonene could be achieved by using a heterogeneous catalyst that is highly active and selective and does not require oxygen removal.

Biomass hydrogenation reactions where a molecule present in this raw material reacts with hydrogen to saturate C═C bonds have a lot of use in the processes of saturation of oils and fats, since linear alkane chains are desirable in some products of the food industry, such as margarines and cocoa fat substitutes. The obtaining of linear hydrocarbons from triacylglycerides and their fatty acid derivatives requires the consecutive hydrogenation of the —COOH groups until the C—O bonds are broken. These reactions require more severe chemical process conditions, such as high pressure, temperature, and long reaction time. The use of a catalyst in these processes is essential, as they are favored in the presence of catalytically active phases that allow H₂ to be activated so that it can be added to the molecules derived from fatty acids. In this sense, the use of highly active and selective catalysts is a fundamental key to the implementation of bioenergy in contemporary societies.

The first generations of heterogeneous catalysts for biomass HDO processes were based on those existing for other hydrotreatment processes, such as those used in sulfur removal and which use MoS₂ as the active phase [Catal. Today, 164 (2011) 533-537], Ni—Mo supported on alumina oxide and titania [MX/a/2018/011787]; while other catalysts are based on precious metals and high costs such as Pt, Pd, among others. Early studies showed that these catalysts were active in removing oxygen contained in biomass derivatives, as it was envisioned as a hydrogenation reaction. However, this type of catalyst is deactivated very quickly and is not resistant to impurities present in the biomass [Appl. Catal. A, 523 (2016) 159-170], which makes the production of oxygen-free hydrocarbons inefficient and requires purification processes of the triacylglyceride matrix. In addition, the water present in the biomass or formed during HDO causes metals to leach out, taking them to the liquid phase, which in the long run causes them to lose their activity, as well as the formation of carbon on the surface is very large. In view of these disadvantages, there is a great need to develop new catalytic materials specifically designed for biomass processing, which avoid sulfides and noble metals as active phases [Renew. Sustain. Energy Rev., 77 (2017) 1375-1384].

Some methods for the transformation of triacylglyceride or methyl ester derivatives or fatty acids have been reported. In most of them, the use of catalysts to reduce the oxygen content of the compounds present in vegetable oils or animal fats stands out. The U.S. Pat. No. 8,026,401 B2 describes a process where two stages are performed to achieve more quantitative hydrodeoxygenation products. Different catalysts are used in each stage, demonstrating the need to use highly active and selective catalysts to be able to perform reactions in one step, which represents a major challenge. In some cases, such as in the international patent application WO 2015/147755 A1, improvements have been made to existing catalysts, through the addition of noble metals to favor the removal of oxygen atoms contained in the molecules derived from biomass, in this case, the improvement of the catalytic materials used in the process increases their activity. The addition of noble metals as an improvement to existing catalysts has been seen as an alternative to increase activity and selectivity towards deoxygenation hydrocarbons from biomass, as claimed in the international patent application WO 2014/018591 A1. However, using noble metals such as Pt makes the process significantly more expensive, as well as causing a negative impact on the environment.

As can be seen in the aforementioned inventions, there is no evidence of the preparation or use of heterogeneous catalytic material based on molybdenum oxide that has an application towards the technical field of this document, so it notoriously exceeds what is known to the applicant, since the change of active phase is very attractive for its industrial application. This represents an advantage in hydrogenation and dehydrogenation processes of compounds present in oils and fats of vegetable and animal origin for the production of linear hydrocarbons with a more sustainable process committed to the environment.

Therefore, one of the aims of the present invention is to provide a heterogeneous catalytic material based on molybdenum oxide for the transformation of compounds present in biomass as derivatives of triacylglycerides and terpenes obtained from vegetable oils or animal fats, which in some cases may come from municipal waste.

Another additional objective is to provide the synthesis of a sulfur-free catalytic material that is highly active and selective due to a correct relationship between active metals, heat treatments and reduction conditions.

Yet another aim is to provide an application of the heterogeneous catalytic material based on molybdenum oxide in hydrogenation or dehydrogenation reactions with high conversions and selectivity to desired products.

### BRIEF DESCRIPTION OF THE DRAWINGS OF THE INVENTION

In order to provide clarity in the description of heterogeneous catalytic material based on molybdenum oxide and its use in biomass transformation processes, which is the subject of this invention, reference shall be made to the accompanying drawings, without limiting the scope of the present invention:

FIG. 1 shows the properties of the molybdenum-based catalyst. (A) Programmed temperature reduction thermogram, (B) Molybdenum XPS and (C) Nickel XPS.

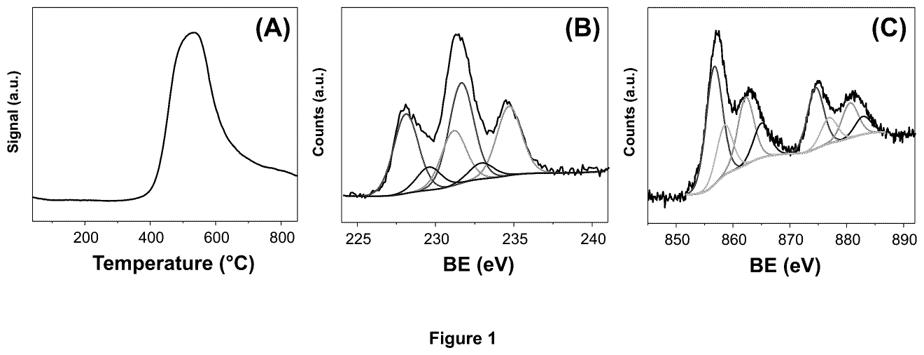

### DETAILED DESCRIPTION OF THE INVENTION

In this invention, metal oxides are used, which are combined in different proportions to obtain heterogeneous catalysts, which produce a conversion of 100% of the compounds present in the biomass with a high selectivity towards desired products. Importantly, the catalytic materials of this invention do not require their pre-activation with sulfur, which is part of a new generation of catalysts for biomass. The use of these materials allows them to operate in combined biomass transformation reactions where it is possible to maintain reaction conditions at lower temperatures and pressures than those used in catalysts of other reports, nor do they require feeding from sulfur sources to artificially maintain their catalytic performance. They have advantages that make them very attractive to obtain high yields of desired products for advanced automotive and aviation fuels, as well as linear hydrocarbons useful for use as additives, lubricants, among others.

In particular, the present invention relates to the synthesis of heterogeneous catalysts using transition metal oxides, as active phases and/or metals as promoters, and supported in silicon oxide (SiO₂) for use in hydrogenation and dehydrogenation processes of chemical compounds present in biomass. SiO₂ is quite attractive for its application in biomass transformation processes, for example, its behavior in aqueous environments allows its adsorbent capacity to be exploited while having greater chemical stability compared to other heterogeneous catalyst supports. In addition, the use of oxides as active phases also allows for heterogeneous catalysts synthesized specifically for this field. The stability of the active phases, their synthesis, handling and use under chemical reaction conditions of bio-based compounds makes the oxide-based active phases an important attraction for the new generation of catalysts in this area.

The synthesis of the catalysts was done by using MoO₃ as the active phase with a composition between 6-20 wt. % and transition metal promoters of non-noble metals, which are in period IV of the periodic table, were used. The weight percentages of the promoters were made between 0.5-5.0 wt. %. The catalyst support is prepared using silica gel, which is calcined between 673.15-873.15 K for 2-8 h in a static muffle with an air atmosphere. Under these conditions the silica gel is converted to amorphous SiO₂, which is used for the preparation of catalysts. The catalysts are prepared by the incipient impregnation method using an aqueous solution containing MoO₃ or ammonium heptamolybdate. Impregnation deposits the active phase on the surface of the silica. This material is calcined in an air atmosphere at a temperature of 773.15 K for 4 h, allowing molybdenum oxide to form on the surface of the catalyst. Subsequently, an aqueous solution containing a nitrate or acetate of a transition metal of period IV is impregnated, which is added to the previously prepared material. The calcination is performed again under the same conditions as MoO₃. It is worth mentioning that catalysts can also be prepared by means of a single step, that is, the molybdenum salts are dissolved in the same solution with those of the nitrates or acetates of the transition metals and impregnated into the support. The two materials can be used as catalysts with very similar catalytic activity between both impregnation methods. The material prepared at this stage is treated in a reducing atmosphere to produce the catalyst. The reduction atmosphere contains H₂ in a purity percentage between 80-100% and the reduction flow is kept constant between 0.5-200 mL/min and is subjected for an interval that can range from 30 min to 8 hours. These final materials are used as heterogeneous catalysts in the transformation of compounds present in biomass.

This invention focused on the application of heterogeneous catalysts based on molybdenum oxide in two reactions of industrial relevance and that can be complementary. In (i) the HDO of triacylglyceride components such as fatty acids, which react with hydrogen in the presence of a heterogeneous catalyst to form linear paraffins, and in (ii) the hydrogenation-dehydrogenation of terpenes that form cyclic hydrocarbons whose properties allow them to be used as polymer precursors or as bio-based aviation fuel.

The catalysts were tested in the HDO of palmitic acid (AP) using a solution of between 0.1-2 wt. % dissolved in n-dodecane. In each test, 0.1 g of the catalyst per 40 ml of solution was used. The tests were carried out in a stainless-steel batch reactor, using hydrogen with a purity of 99.99% at a pressure between 0.68-6.89 MPa. The temperature for HDO with these catalytic materials was between 523-673 K with constant agitation between 100-500 rpm. The catalysts were evaluated for 6 h and the analysis of conversion and composition of products was carried out by means of gas chromatography coupled to mass spectrometry. The reaction scheme of the fatty acid HDO that takes place on the catalysts within this invention is shown in Diagram 1:

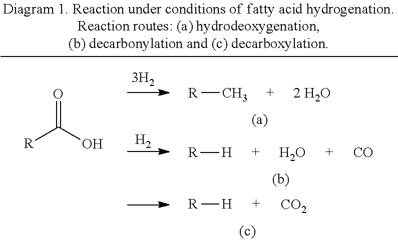

This process can be carried out through three very well differentiated reactions: (a) hydrodeoxygenation, (b) decarbonylation and (c) decarboxylation. The catalyst used can favor one route over the others. The desired selectivity is towards the production of oxygen-free hydrocarbons without carbon loss, which can be used in mixtures with hydrocarbons obtained from fossil sources and allows reducing their carbon footprint. Importantly, the catalysts with higher activity are not necessarily highly selective and the presence of oxygenated compounds in pathway (a) can convert acids into alcohols, compounds that still contain oxygen atoms in their structures, as shown in Diagram 2 with the three molecules of H₂ in the reactions explicitly, and the formation of water as a by-product:

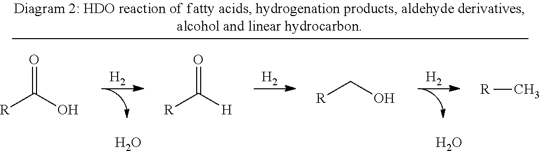

The results of the catalysts testing in HDO were obtained based on the conversion of palmitic acid (PA), as well as the percentage of linear hydrocarbons formed, which represent the percentage of chemicals without oxygen atom content in their structure, i.e. complete HDO.

Limonene was used for the catalytic activity tests of terpene-derived cyclic compounds. The chemical structure of this compound allows it to undergo hydrogenation and dehydrogenation reactions, the reaction scheme is shown in Diagram 3. For the first case, partial or total hydrogenation is attractive, while for the second case dehydrogenation is useful to form aromatic compounds. Partial hydrogenation makes it possible to produce compounds that serve as monomers of high value-added polymers, while total hydrogenation produces compounds that could be used as aircraft fuel. On the other hand, dehydrogenation reactions serve to produce aromatic compounds that could allow them to be added as a component or additive to jet fuel. In addition to this and most attractive for its application, is the combination of HDO with the dehydrogenation of limonene in a single step. By doing this, two things of great importance are achieved: (i) reducing the consumption of H₂ in the HDO and (ii) adding aromatic compounds to the bio-based jet fuel. For the hydrogenation reactions, temperatures ranging from 373.15-613.15 K were used, as well as pressure intervals ranging from 0.34-6.89 MPa. In hydrogenation reactions, H₂ was used as atmosphere, while in dehydrogenation reactions, an inert gas is used, which can be N₂ or Ar.

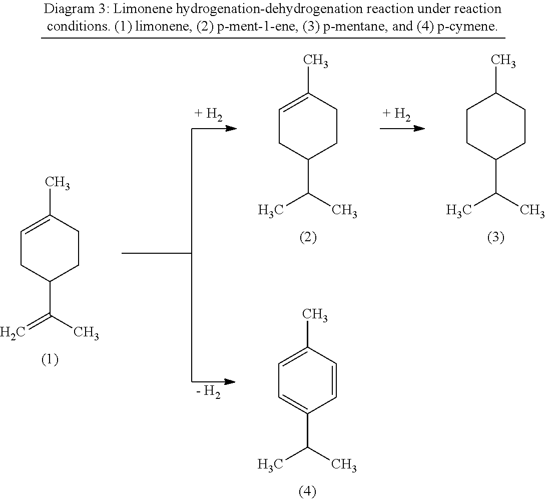

### EXAMPLES

With the general data of this invention, some examples are described below that show the characteristics and performance of the new catalytic materials applied to biomass transformation processes that, of course, only have an illustrative purpose and are not limited to the technical scope of the present invention.

### Example 1. Preparation of Heterogeneous Catalytic Materials and their Application in the HDO of Palmitic Acid

The catalyst support is prepared using silica gel, which was calcined at 823.15 K for 4 h in a static muffle with an air atmosphere. The material after calcination was used as support for the heterogeneous catalysts.

The catalysts are prepared by the incipient impregnation method using an aqueous solution of ammonium heptamolybdate to obtain 12 wt. % of MoO₃, which is used as the active phase. This material is calcined under air atmosphere at a temperature of 773.15 K for 4 h, allowing molybdenum oxide to form on the surface of the catalyst. Subsequently, an aqueous nitrate solution of a transition metal of period IV, such as Fe, Ni or Cu, was impregnated at 3.5 wt. %, which was added to the previously prepared material. The calcination is performed under the same conditions. Single-step impregnation of both metals produces the same result in material properties and catalytic performance. The material is then treated in a reducing atmosphere from H₂ to 673.15 K, at atmospheric pressure to produce the catalyst.

The results of the textural characteristics of these materials, as well as their catalytic performance in the HDO of palmitic acid are shown in Table 1. The best catalytic material based on molybdenum oxide, which contains nickel, exhibited 100% conversion of palmitic acid, as well as 93% linear alkane formation.

The data show that molybdenum oxide without the addition of a metal as a promoter has a catalytic activity and selectivity of up to 15 and 5%, respectively. The use of nickel and copper as promoters of the hydrodeoxygenation reaction stands out, with nickel contributing to the obtaining of a catalytic material with the highest activity and selectivity in the conditions of hydrodeoxygenation, and which will be referred to hereinafter as IMP-BIO1.

### Example 2. Properties of the IMP-BIO1 Catalyst

The catalytic material prepared with nickel-molybdenum, in Example 1, is called IMP-BIO1. This material was characterized by means of programmed temperature reduction (TPR) and X-ray photoelectronic spectroscopy (XPS) techniques. These two techniques seek to complement the information on the properties of this material, in addition to the surface area of Table 1. The profile of the reduction reaction of the material in the oxide state presents a temperature range that is between 673.15 and 873.15 K, with a heating rate between 1-40 K/min, (Diagram 2a), with the maximum reduction peak around 773.15. The XPS spectra for molybdenum and nickel are shown in Diagrams 2b and 2c respectively, these spectra belong to the catalyst after the reduction process described in Example 1. The molybdenum XPS shows the presence of three species very well distinguishable by the position of their binding energies (BE), which can be assigned to three states of molybdenum oxidation in these materials. The integration of the curves allows us to obtain a distribution of the relative concentration as follows: Mo⁶⁺=0.49, Mo⁵⁺=0.11 and Mo⁴⁺=0.40. The XPS for nickel allows you to appreciate a combination of Ni metallic and Ni²⁺ species. This metal is a catalyst promoter and may also have a role as an active site in the metallic state, in this material the Ni⁰ and Ni²⁺ species coexist.

### Example 3. Preparation Conditions in the Material Reduction Stage

The molybdenum-nickel catalytic material was studied under different reduction temperatures in the final stage of its preparation. This material reduction temperature was varied with intervals of 100 K for each material and its performance in the HDO was tested, keeping all other variables fixed. The results of the catalytic activity as a function of the reduction temperature in the catalyst preparation are shown in Table 2. The reduction ramps provide different catalytic materials, obtaining an optimal temperature for synthesis between 573.15-773.15 K, since the conversion at 6 h of reaction is 100% and the formation of deoxygenated compounds is greater than 84%.

The material reduction step is important to increase the activity and selectivity of the developed catalyst. It can be observed that the catalytic material without the reduction step presents the least activity and selectivity in the HDO reaction. It is observed that the increase in the reduction temperature of the catalytic material allows the conversion of the AP, as well as the production of oxygen-free hydrocarbons.

### Example 4. Effect of H₂ Pressure on the Performance of the IMP-BIO1 Catalyst

The variation of H₂ pressure in the HDO process is also shown as an example of the versatility of the IMP-BIO1 catalyst, since a low-pressure process of H₂ is desirable but also to obtain a high conversion of the HDO reaction. Three pressure values of H₂ were used during the HDO of palmitic acid on the catalyst reduced to 673.15 K, the results of the HDO as a function of the pressure at 6 h of reaction are listed in Table 3. In the three pressures used, a conversion of 100% and a deoxygenation percentage of more than 90% is achieved. However, the best catalytic activity was obtained with a pressure of 3.45 MPa since the conversion is 100% and the deoxygenation is total as no by-product with oxygen is detected in its structure. A decrease in pressure to perform HDO means a reduction in H₂ gas costs, which is very feasible with this IMP-BIO1 catalyst.

The high activity and selectivity of this catalyst at low H₂ pressures is of utmost importance, because most commercial hydrotreating catalysts require an H₂ pressure in the order of or greater than 5.17 MPa. Thus, IMP-BIO1 catalytic material is suitable in the reaction of HDO of PA at low pressure.

### Example 5. Limonene Hydrogenation

The IMP-BIO1 catalyst was tested in the hydrogenation reaction of limonene, this terpenoid can exhibit different degrees of hydrogenation in the reaction products. On the other hand, the hydrogenation conditions of this terpene require that they be moderate, since it is the hydrogenation of C═C bonds in cyclic hydrocarbons. The results of limonene hydrogenation on the IMP-BIO1 catalyst as a function of the reaction temperature are shown in Table 4. The reaction conditions were at 1.03 MPa of H₂, with constant stirring, 4 h of reaction and with temperature variation from 373.15 to 573.15 K.

The conversion of limonene at a temperature of 373.15 K and reaction time of 4 h reaches 85%. However, the increase in the hydrogenation reaction temperature allows a greater conversion of limonene to 100% from 473.15 K. It is important to note that the reaction temperature also improves the selectivity towards the production of aromatic compounds (compound 4 of Diagram 3). Indeed, the relationship between the aromatic cycle and the non-aromatic cycle increases as the reaction temperature rises, which can be important, since aromatic compounds could be fed to the products of the HDO. The reaction performed at 573.15 K resulted in a ratio of the aromatic cycle (4) to the aliphatic cycle (3) of 0.85.

### Example 6. Limonene Dehydrogenation

The IMP-BIO1 catalyst was also tested in the limonene dehydrogenation reaction, and it allows the synthesis of compound 4, FIG. 3. In this process, an inert atmosphere of N₂ at a pressure of 1.03 MPa was used. The inert atmosphere was chosen to promote the dehydrogenation reaction because of temperature and the presence of the catalyst, which is active under these conditions. The conversion results, as well as selectivity by the ratio of compounds (4)/(3) in FIG. 3 are shown in Table 5.

The IMP-BIO1 catalyst showed a total conversion of limonene from 523.15 K, with a high preference for the formation of the aromatic compound. The selectivity towards the aromatic product with respect to the aliphatic is around 303.15 to 573.15 K.

TABLE 1
Textural properties of catalysts based on M-MoO₃/
SiO₂ and catalytic performance in 6 h of HDO.

| | | | Formation of |
|---|---|---|---|
| | S_{BET} | Palmitic acid | linear alkanes |
| M | (m²/g) | conversion (%) | (%) |
| — | 350 | 15 | 5 |
| Fe | 324 | 20 | 5 |
| Ni | 301 | 100 | 93 |
| Cu | 299 | 69 | 21 |

TABLE 2
Catalytic activity results in 6 h of HDO as a
function of the reduction temperature
in the heterogeneous catalyst preparation.

| Reduction | Palmitic Acid | Formation of linear |
|---|---|---|
| Temperature | Conversion | alkanes |
| (K) | (%) | (%) |
| Oxide | 26 | 32 |
| 373.15 | 25 | 32 |
| 473.15 | 25 | 40 |
| 573.15 | 100 | 84 |
| 673.15 | 100 | 93 |
| 773.15 | 100 | 98 |

TABLE 3
Results of catalytic activity as a function
of the H₂ pressure used in the HDO at 6 h.

| H₂ Pressure | Palmitic acid | Formation of |
|---|---|---|
| (MPa) | conversion (%) | linear alkanes (%) |
| 1.03 | 100 | 91 |
| 3.45 | 100 | 100 |
| 5.17 | 100 | 93 |

TABLE 4
Results of catalytic activity as a function
of reaction temperature in limonene
hydrogenation at 4 h. Ratio of compounds
of Diagram 3, as an estimation of selectivity.

| Reaction | Limonene | Compound |
|---|---|---|
| temperature | Conversion | ratio |
| (K) | (%) | (4)/(3) |
| 373.15 | 85 | 0 |
| 473.15 | 96 | 0.08 |
| 573.15 | 100 | 0.24 |
| 673.15 | 100 | 0.46 |
| 773.15 | 100 | 0.85 |

TABLE 5
Catalytic activity results as a function of
reaction temperature in limonene dehydrogenation
at 4 h. Relationship of compounds of the
Diagram 3, as an estimation of selectivity.

| Reaction | Limonene | Compound |
|---|---|---|
| temperature | Conversion | ratio |
| (K) | (%) | (4)/(3) |
| 373.15 | 0 | ND |
| 473.15 | 25 | ND |
| 573.15 | 82 | 3 |
| 673.15 | 100 | 29 |
| 773.15 | 100 | 30 |

1. A heterogeneous sulfur-free catalyst for the hydrogenation and dehydrogenation of compounds in biomass, characterized by comprising, between 6-20 wt. % molybdenum oxide, between 0.5-5.0 wt. % of a promoter of the transition metals of period IV of the periodic table selecting Fe, Ni or Cu, and supported between 75-93.5 wt. % of silica.

2. The heterogeneous catalyst, in accordance with claim 1, is characterized by the nitrogen physisorption with surface area between 290 and 350 m²/g; in powder form with the possibility of extrusion into cylindrical form.

3. The heterogeneous catalyst, in accordance with claim 1, is characterized by the following chemical properties: between 6-20 wt. % of MoO₃ and with transition metal promoters of non-noble metals, which are in period IV of the periodic table. The weight percentages of the promoters between 0.5-5.0 wt. %.

4. The heterogeneous catalyst, in accordance with claim 1, is characterized by the following catalytic properties: between 50-100% fatty acid conversion, between 32-100% formation of linear oxygen-free alkanes, limonene conversion between 85-100% of limonene in hydrogenation processes and between 35-100% of limonene conversion in dehydrogenation processes.

5. A method for preparing the catalyst of claim 1, characterized by the following steps: (a) calcining the catalyst support by selecting silica gel at 823.15 K for 4 h in a static air atmosphere; (b) prepare the catalyst by the incipient impregnation method using an aqueous solution of ammonium heptamolybdate to obtain 5-20 wt. % of MoO₃ as the active phase; (c) calcinate the material obtained in subparagraph (b) in an air atmosphere at a temperature of 773.15 K for 4 h; (d) subsequently, impregnate the product obtained in subparagraph (c) with aqueous nitrate solution of a transition metal of period IV, selecting Fe, Ni or Cu, at 3.5 wt. %; (e) repeat calcination under the same conditions with the material previously obtained; f) reduce the material obtained in an atmosphere of H₂ between 573.15-873.15 K, preferably to 673.15 K at atmospheric pressure to produce the catalyst.

6. The method, in accordance with claim 5, is characterized by the incipient impregnation of metals in one or two stages until the manufacture of the catalytically active species by their heat treatment in a hydrogen atmosphere.

7. An HDO process using the catalyst of claim 1, characterized by the following steps: (a) loading the catalyst into a batch reactor; (b) to carry out a thermal pretreatment of said catalyst under constant flow of hydrogen between 0.5-500 mL/min and a temperature range between 573.15-873.15 K; c) contact said pre-treated catalyst with biomass-derived compounds by selecting free fatty acids, triacylglycerides, simple lipids, compound lipids and terpenoids under the following operating conditions: a temperature range between 453.15-693.15 K; pressure between 0.10-13.79 MPa; d) produce a 100% conversion to oxygen-free hydrocarbons with a selectivity between 30-100%.

8. The process, in accordance with claim 7, is characterized by the fact that it has an active performance of a 100% conversion of terpenes, such as, limonene and selective to obtain the compounds p-mentane and p-cymene.

9. The process, in accordance with claim 7, is operated in a temperature range between 323.15-623.15 K and a pressure range between 0.01-4.83 MPa.

10. The process, in accordance with claim 7, converts more than 95% limonene to the aromatic compound p-cymene
